(19) United States
(12) Patent Application Publication
(10) Pub. No.: US 2019/0307227 A1
(43) Pub. Date: Oct. 10, 2019

(54) Fresh Cosmetic Composition Delivery System

(71) Applicant: ELS MANAGEMENT LLC, Melville, NY (US)

(72) Inventors: Janet Pardo, New York, NY (US); Robert CRESCAS, Madison, NJ (US); Christopher POLLA, Massapequa, NY (US); Lorraine DRAKE, New York, NY (US); Thomas OWEN, Montauk, NY (US)

(21) Appl. No.: 16/470,169

(22) PCT Filed: Dec. 19, 2017
(86) PCT No.: PCT/US2017/067195

§ 371 Date: Jun. 14, 2019

(30) Foreign Application Priority Data
Dec. 20, 2016 (US) 15384829

(51) Int. Cl. A45D 34/04; B65D 81/32; B65D 83/00; A61K 8/67; A61Q 5/10; A61Q 19/00

(52) U.S. Cl. CPC A45D 34/04; B65D 81/3211; B65D 83/0005; A45D 2034/005; A61K 8/671; A61Q 5/10; A61Q 19/00; A61K 8/676

(57) ABSTRACT

A fresh composition delivery system includes a package with two compartments separated by a foil seal for separating a cosmetically acceptable carrier from an unstable active ingredient. An elastomeric bulb with a dart can be actuated by the user to pierce the foil seal so that the carrier and the active can be mixed in the package to form a composition shortly before use. For the resulting mixed composition, the level of the unstable active ingredient in the composition decreases by less than 6% over 7 days when stored at 25° C. at 60% relative humidity.

### FIELD OF THE INVENTION

The present invention relates to a fresh cosmetic composition delivery system. In particular, the present invention is directed to a package and composition for dispensing an otherwise unstable active.

### BACKGROUND OF THE INVENTION

Many cosmetically active ingredients are unstable. For example, Vitamin C when formulated into a cosmetic vehicle is known to provide clinical benefits such as improved tone, reduced lines/wrinkles, and improved firmness. Vitamin C is known to be stable in dry, solid, crystalline form. However, Vitamin C is also known to degrade in aqueous vehicles. This invention is intended to keep a stable Vitamin C powder separate from, for example, an aqueous vehicle until just before use. In this way, Vitamin C in an aqueous vehicle can be kept stabile for at least 7 days, an appropriate time period for the intended use of the product. The present invention is intended to keep an unstable active ingredient separate from the dermatologically acceptable carrier until shortly before use, and provide the user with a mixed composition (active and carrier) having a level of the unstable active ingredient in the composition that is efficacious and usable by a consumer for at least a seven days.

### BRIEF SUMMARY OF THE INVENTION

A main object of the invention is to provide a package and a composition for dispensing an otherwise unstable active in a ‘fresh’ condition for at least 7 days.

A package is used for separating a first and second component of a cosmetic composition. The first component is a cosmetically acceptable carrier. The second component is an unstable active ingredient. The unstable active ingredient can be any that is subject to degradation by light, oxidation, or by combination with other ingredients. The package comprises a tube with a reservoir for the first component and a cartridge in the tube and in fluid communication with the reservoir. The cartridge has a chamber for isolating the second component, the unstable active ingredient, from the first component. In this way, the unstable active ingredient is maintained in an isolated, stable condition until it is mixed with the carrier to form the composition prior to use. In the resulting mixed composition, the level of the unstable active ingredient in the composition decreases by less than 6% over 7 days when stored at 25° C. at 60% relative humidity.

### BRIEF DESCRIPTION OF THE DRAWINGS

FIG. 1 is a top, front perspective view of a package suitable for use with the system of the invention.

FIG. 2 is a top, front perspective view of the package of FIG. 1.

FIG. 3 is a front elevation cross sectional view of the package of FIG. 1.

FIG. 4 is a front elevation cross sectional view of the package of FIG. 1 with the dart moved to pierce the foil seal.

FIG. 5 is an enlarged partial view of the front elevation shown in FIG. 3.

FIG. 6 is a bottom plan view of a cartridge suitable for use in the package illustrated in FIG. 1.

FIG. 7 is a top plan view of the cartridge illustrated in FIG. 6.

FIG. 8 is a top, front perspective view of the cartridge illustrated in FIG. 6.

FIG. 9 is a front elevation cross-sectional view of the cartridge illustrated in FIG. 6.

FIG. 10 is a front elevation cross-sectional view of an alternative embodiment of the cartridge illustrated in FIG. 6.

### DETAILED DESCRIPTION OF THE INVENTION

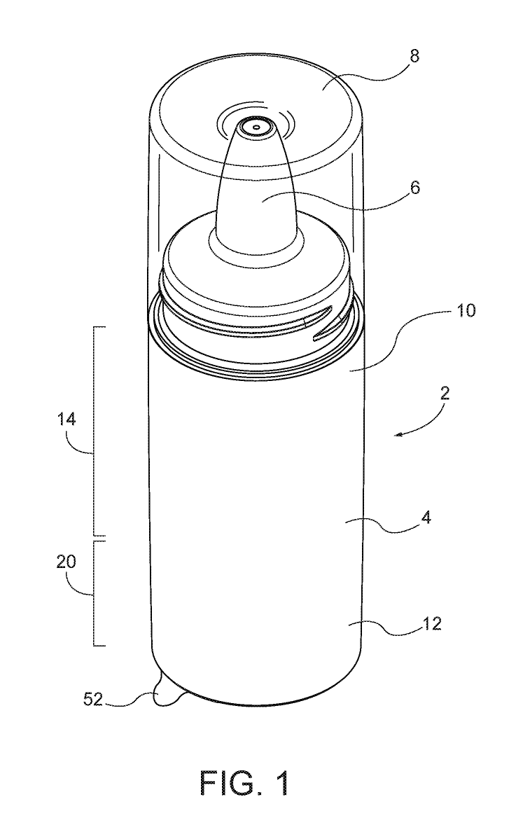

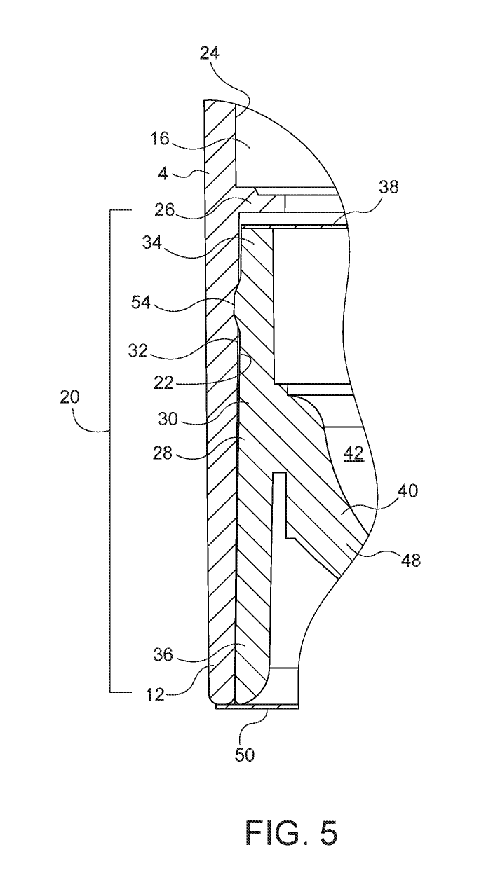

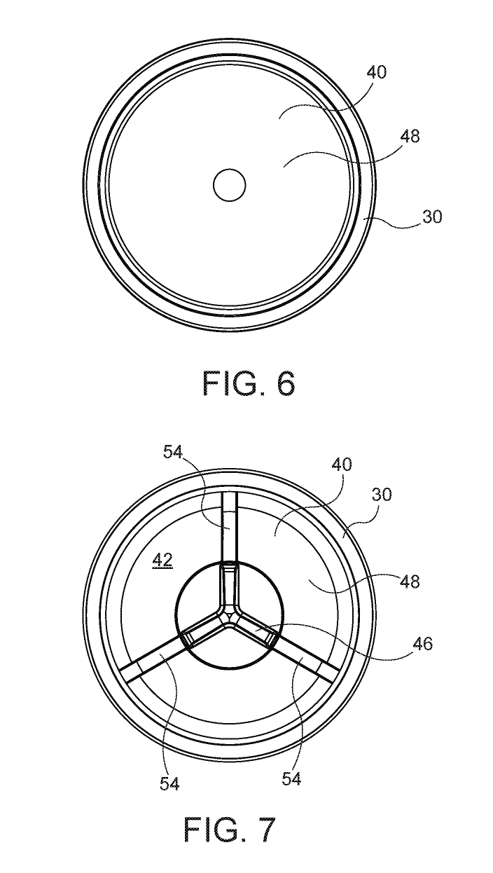

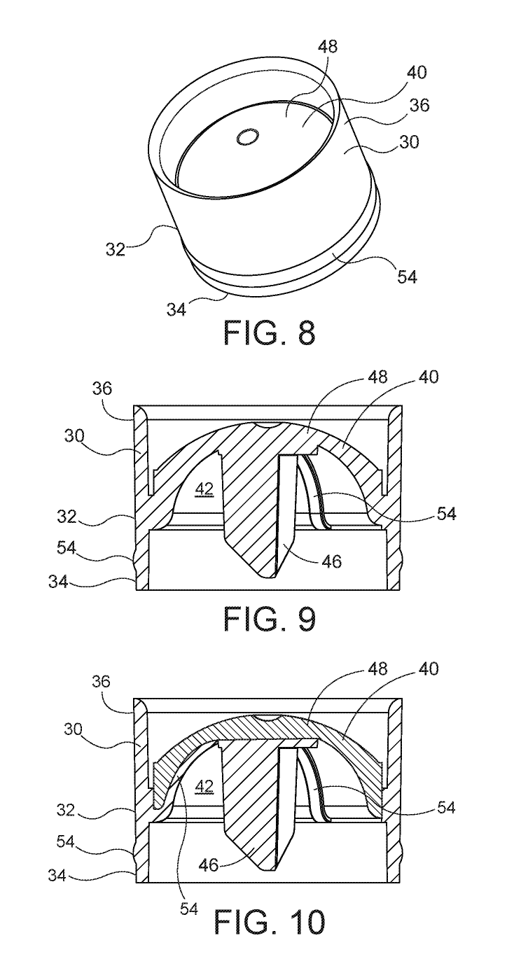

Referring now to FIGS. 1-10, a package 2 for an exemplary fresh composition delivery system comprising a composition and the package 2 is illustrated. The composition comprises a first component including a dermatologically acceptable carrier. The dermatologically acceptable carrier may include a liquid or serum as described in further detail below. The composition further comprises a second component including at least one unstable active ingredient. The unstable active ingredient may be any skincare active ingredient that is unstable and subject to degradation when exposed to light, oxidation, or by combination with other ingredients. Examples of unstable active ingredients include, for example, Vitamin A, Vitamin C, Vitamin E, oxidation susceptible botanicals, etc. Further examples are provided below.

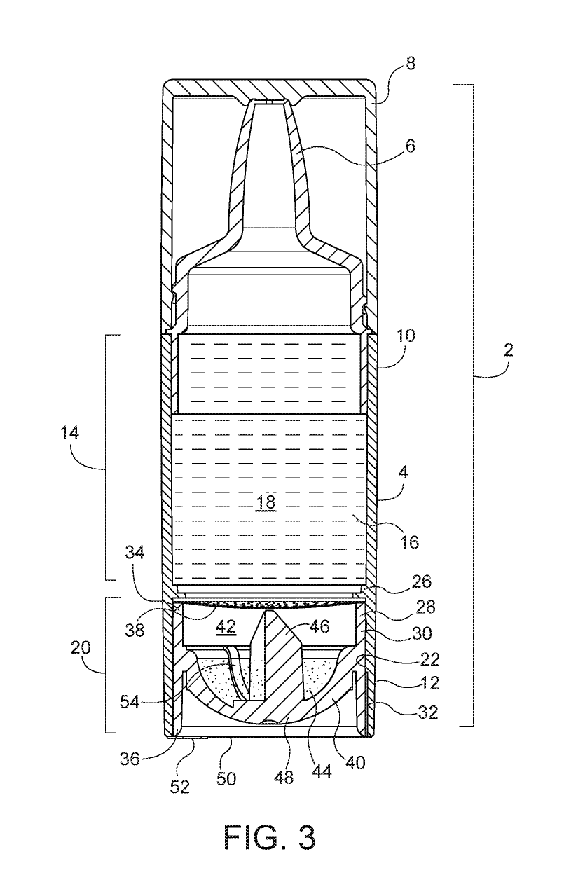

The package 2 is adapted for keeping the first component and second component of the composition separate before use. The package comprises a tube 4 having a dispensing spout 6 with a cap 8 at a first end 10 and an open second end 12. The tube has an inner surface 24. A first portion 14 of the tube adjacent the first end 10 defines a reservoir 16 (see FIG. 3) between the spout 6 and a bulkhead 26 located on the inner surface 24 between the first portion 14 and the second portion 20 of the tube 4. A second portion of the tube 20 adjacent the second end 12, and between the bulkhead 26 and the second end 12, defines on the inner surface 24 an inwardly directed sealing surface 22.

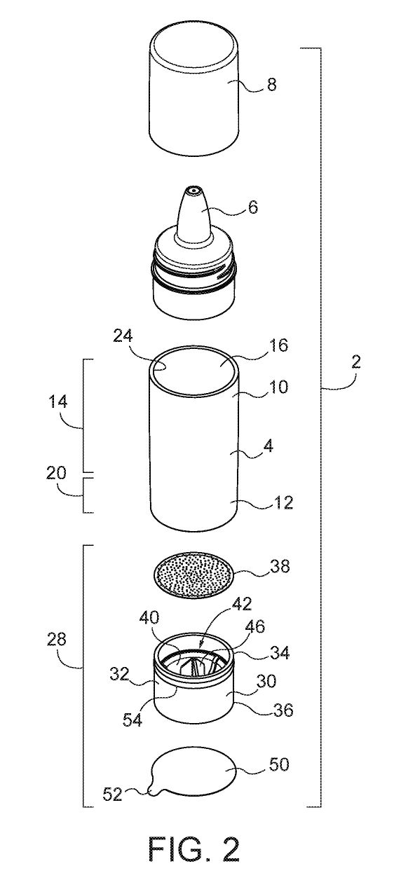

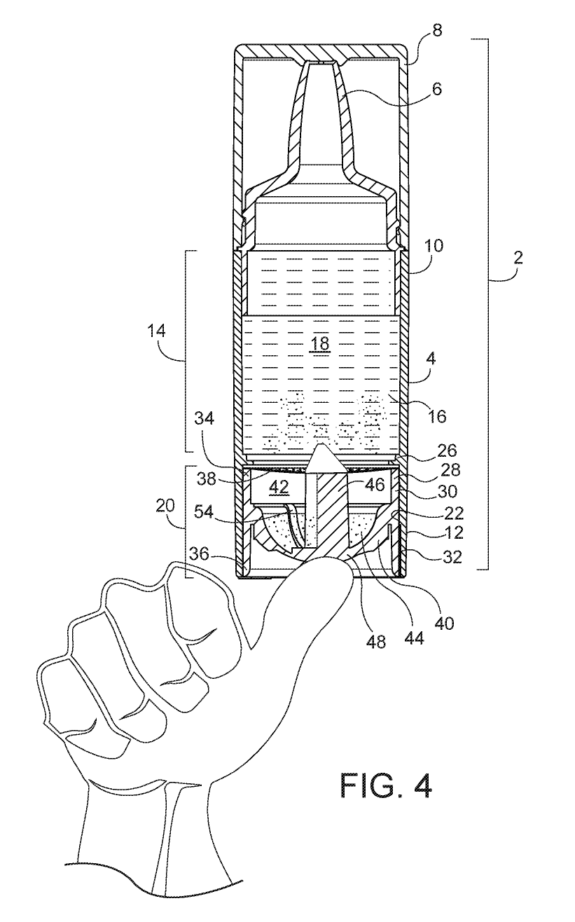

As best illustrated in FIGS. 2-5, a cartridge 28 is provided having a tubular body 30 dimensioned to fit closely in the second portion 20 of the tube 4. The body 30 of the cartridge 28 has an outwardly directed sealing surface 32 for engaging the inwardly directed sealing surface 22 of the tube 4 in airtight engagement. A circumferential rib 54 on the outwardly directed sealing surface provides additional frictional pressure to secure and seal the cartridge 28 in the second portion 20 of the tube 4. An inner end 34 of the body 30 of the cartridge 28 is positioned in the tube 4 such the inner end 34 is directed toward and openable to the reservoir 16. The body 30 of the cartridge 28 has an outer end 36 opposite the inner end 34. As illustrated in FIGS. 3 and 4, the outer end 36 of the body 30 of the cartridge 28 is proximal to and in alignment with the open second end 12 of the tube 4. However, the outer end 36 may also be recessed within the second end 12 of the tube 4, or alternatively extend out from the second end 12 of the tube 4. A foil inner seal 38 closes the inner end 34 of the body 30 of the cartridge 28. With the cartridge secured in the second portion 20 of the tube 4, the foil inner seal 38 also forms an end of the reservoir 16 of tube 4. With the cartridge 28 secured in the second portion 20 of the tube 4, a quantity of the first component 18 in liquid form, the cosmetically acceptable carrier, is stored in the reservoir 16.

An actuator membrane 40 is located in the body 30 of the cartridge 28 approximately mid-way between the inner end 34 and the outer end 36. The actuator membrane 40 forms a hermetic barrier between the inner end 34 and the outer end 36 of the body 30. A hollow chamber 42 is defined in the body 30 of the cartridge 28 between the foil inner seal 38 and the actuator membrane 40. A quantity of the second component 44, i.e., the unstable active ingredient, is stored in the chamber 42. In FIGS. 3 and 4, the second component 44 is illustrated as granular in form, but it may be in any suitable form, such as, for example, liquid, powder or one or more solid tablets (not shown). A blade or dart 46 is mounted on the actuator membrane 40 such that it projects into the chamber 42 toward the foil inner seal 38. As illustrated in FIG. 4, the actuator membrane 40 is selectively movable toward the foil inner seal 38 on the inner end 34 such that the blade or dart 46 pierces the foil inner seal 38 to release the quantity of the second component 44 into the quantity of the first component 18. This allows the first and second components, 18 and 44, respectively, to mix and form the final composition shortly before dispensing and use.

After mixing, the composition so formed by mixing of the first and second components, 18 and 44, respectively, in the reservoir 16 has a level of the unstable active ingredient (second component 44) that decreases by less than 6% over 7 days when stored at 25° C. at 60% relative humidity.

The actuator membrane 40 may be an elastomeric bulb 48 (also referred to herein as an actuator bulb or actuator button or actuator) projecting convexly toward the outer end 36. The elastomeric bulb 48 acts as an actuator button for moving the blade or dart 46 to pierce the foil inner seal 38. The outer end 36 of the body 30 of the cartridge 28 may be closed by a foil outer seal 50 that keeps dust or other debris from entering the outer end 36 of the cartridge 28. For the convenience of the user, the foil outer seal 50 may be provided with a tab 52 projecting radially outwardly from the foil outer seal (as illustrated in FIGS. 1 and 2) to facilitate removal of the foil outer seal 52 from the outer end of the cartridge. Preferably the tab 52 is folded flat against the seal 50 as illustrated in FIG. 3 to present a tidy appearance and to avoid premature removal of the seal.

This invention is intended to be able to utilize different unstable active ingredients and molecules and in combination with different dermatologically acceptable carriers or vehicles. For example, in the case of Vitamin C (ascorbic acid), an amount from 1-20% may be used. In the case of Vitamin A (retinol), an amount from 0.01-2.7% may be use.

The unstable active ingredient comprising the second component may be any ingredient that is subject to degradation by light, oxidation, time, pH extremes or by combination with other ingredients. The unstable active ingredient may be any ingredient that would benefit from being isolated from the rest of the formula making up the full composition. For example, the unstable active ingredient may be Vitamin C, Vitamin E, oxidation susceptible botanicals, retinol, resveratrol (or other stilbenoids), tocopherols, retinoids, folic acid or hair dye. The unstable active ingredient may be a caffeic, chlorogenic, or gallic acid that is not stable at high pH. The unstable active ingredient may be an oil that is vulnerable to light or oxidation, such as, for example, high polyunsaturated oils, high linolenic acid oils, flax seed oil, raspberry seed oil, cranberry seed oil, black current seed oil, Sysimbrium oil, Perilla seed oil, Camelina sativa, Salvia hispanica, high linoleic oils, pomegranate seed oil, Prunus ameniaca (apricot seed kernel) oil, Juglans regia (walnut) oil, hemp seed oil or wheat germ oil. The unstable active ingredient may be provided in the chamber in a powder, liquid, tablet or cake form.

The first component 18 including the dermatologically acceptable carrier may be a liquid having a formula according to the following Example 1.

| Raw Material | Function | % |
|---|---|---|
| Purified Water | vehicle | 76.300 |
| Glycerine | humectant | 5.000 |
| Disodium EDTA | chelating agent | 0.050 |
| Carbomer | viscosity modifier | 0.150 |
| Bis-PEG-18 Methyl Ether | aesthetic modifier | 0.550 |
| Dimethyl Silane | | |
| Dimethicone | aesthetic modifier | 4.000 |
| Squalane | aesthetic modifier | 1.750 |
| Alcaligenes Polysaccharides | viscosity modifier/stabilizer | 0.100 |
| Dipropylene Glycol | solubilizer/dispersing agent | 1.000 |
| Xanthan Gum | viscosity modifier/stabilizer | 0.300 |
| Potassium Sorbate | preservative | 0.050 |
| Phenoxyethanol | preservative | 0.630 |
| Tromethamine | pH adjuster | 0.120 |

The first component 18 (carrier) illustrated in Example 1 is suitable for use with a second component 44 (active) including Vitamin C (Ascorbic Acid) at a level of 10% to form the fresh composition. In the forgoing example, 1 gram of Vitamin C is provided in the chamber 42 in the cartridge 28 and 9 ml of cosmetically acceptable carrier 18 is provided in the reservoir 16 in the tube 4. Mixing the two components yields a supply of ‘fresh’ composition suitable for use within 7 days.

An alternative first component 18 suitable for use in the present invention is illustrated in the following Example 2:

| Raw Material | Function | % |
|---|---|---|
| Purified Water | Vehicle | 61.730 |
| Disodium EDTA | chelating agent | 0.050 |
| Bis-PEG-18 Methyl Ether | aesthetic modifier | 0.550 |
| Dimethyl Silane | | |
| Methyl Gluceth-20 | Humectant | 1.320 |
| Glycerine | Humectant | 0.890 |
| Butylene Glycol | solubilizer/dispersing agent | 2.200 |
| Polysorbate-20 | Solubilizer | 2.660 |
| Dimethicone/Polysilicone-11 | aesthetic modifier | 8.700 |
| Methyl Trimethicone | aesthetic modifier | 2.700 |
| Lauryl PEG-9 | Emulsifier | 1.800 |
| Polydimethylsiloxyethyl | | |
| Dimethicone | | |
| Vinyl Dimethicone/Methicone | aesthetic modifier | 2.700 |
| Silsesquioxane Crosspolymer | | |
| Xanthan Gum | viscosity adjuster/stabilizer | 0.180 |
| Carbomer | viscosity adjuster/stabilizer | 0.180 |
| Ammonium Acryloyldimethyl | viscosity adjuster/stabilizer | 0.200 |
| Taurate/VP Copolymer | | |
| Caprylyl Glycol | preservative | 0.180 |
| Sodium benzoate | preservative | 0.090 |
| Phenoxyethanol | preservative | 0.400 |
| Caustic Soda (30% NaOH) | pH adjuster | 0.390 |
| Polysorbate-20 | carrier/dispersant | 7.340 |
| Soybean Oil | carrier | 1.390 |

The first component 18 illustrated in Example 2 is suitable for use with Vitamin A (retinol) containing compound (for example, Retinol 10S sourced from BASF) in an amount of about 2.7% to form the fresh composition. The retinol containing compound is a blend of retinol with soybean oil wherein the typical concentration of retinol is about 11%. Accordingly, the amount of retinol delivered by the system would be about 0.297%.

Of course, it will be understood that the unstable active ingredient (second component 44) may be provided in any suitable amount to provide a wide range of percentage amounts in the final formula and the ingredient percentages of the first component 18 will need to be adjusted accordingly to accommodate less or more unstable active ingredient.

Each part of the package may be made by injection molding or other suitable molding means.

The cartridge 28 containing the unstable active ingredient, second component 44, is preferably sealed at each end by the foil inner seal 38 and the foil outer seal 50. The foil inner seal 38 and foil outer seal 50 may be made from the same material, a 0.15 mm thick foil consisting of 96% aluminum and 4% polyethylene (available under the tradename Amcor Steril Up® Aluthene II), or other suitable materials. A coating or coatings on the foil inner seal is optional. The foil outer seal may have additional coatings or may have indicia printed thereon. Each of the foil inner seal 38 and foil outer seal 50 is preferably secured to the inner and outer ends, 34 and 36 respectively, of the cartridge 28 by induction heating and application of pressure in an atmospherically controlled environment. Alternatively, the seals may be secured with an adhesive. The foil inner seal 38 may be referenced as the “punch through foil” and the foil outer seal 50, which is visible to the consumer, may be referenced as the “tear off foil”. As noted above, the foil outer seal 50, the tear off foil, may be provided with a radially outwardly extending tab 52 (as illustrated in FIGS. 1 and 2), also referred to as a “pull tab” that can be used by the consumer to facilitate access to the actuator bulb 48 in the outer end 36 of the cartridge 28. For esthetic purposes, as illustrated in FIG. 3, the pull tab 52 is preferably folded down against the foil outer seal 50 to present a tidy appearance and to avoid premature removal of the seal.

The body 30 of the cartridge 28 is preferably an injection molded polypropylene plastic with an over-molded TPE actuator membrane in the form of an elastomeric bulb 48 projecting convexly toward the outer end 36 of the body 30 of the cartridge 28. Other materials may be suitable for use with other unstable actives and other carriers. The construction enables the bulb 48 to be compressed by the user, pushing the blade or dart 46 through the foil inner seal 38 and breaking the foil inner seal 38 to allow mixing of the unstable active ingredient of the second component 44 with the cosmetically suitable carrier of the first component 18. The force required to move the actuator bulb 48 to advance the dart 46 to pierce the foil inner seal 38 averages 6.4 lbs within a range of 5.6 lbs. to 7.2 lbs. As illustrated more clearly in FIG. 10, preferably, arms 54 integrally formed with the body 30 of the cartridge 28 from polypropylene plastic extend from the body 30 to support the dart 46. Preferably, the arms 54 are covered by the overmolded TPE bulb 48. The arms 54 enhance the stability of the dart 46 and the function of the elastomeric bulb 48. The arms 54 flex, enabling the dart 46 to move sufficiently to pierce the foil inner seal 38. The thin profile of the arms 54 and the elasticity of the bulb 48 allow the arms and bulb to return to their original resting position. The user is then able to repeat the compression of the bulb 48 to dispense the mixed composition through the spout 6 for application and use.

To assemble the package, the cartridge 28 is provided with the actuator membrane 40 secured to form one end of the chamber 42 in the body 30 of the cartridge 28. A suitable quantity of the second component 44, the unstable active ingredient, is added to the chamber 42 in the cartridge 28, and the foil inner seal 38 is secured to inner end 34 of the body 30 of the cartridge 28 to close the chamber 42. The foil outer seal 50 may also be secured to outer end 36 of the body 30 of the cartridge 28 at this time. A preassembled and decorated tube 4 with a cap 8 secured to the spout 6 is provided at a filling station. After air is evacuated from the reservoir 16 in the tube 4, the reservoir 16 is filled with the first component 18 (the cosmetically acceptable carrier) in liquid form. The cartridge 28 is secured in the second end 12 of the tube 4 by friction or interference fit. The inwardly directed sealing surface 22 of the tube 4 engages the outwardly directed sealing surface 32 of the cartridge 28 in an airtight manner. The cap 8 seals against the tip of the spout 6 preventing leakage until selectively opened by the user.

When the user is ready to use the package 2, the foil outer seal 50 is removed by pulling the pull tab 52. As illustrated in FIG. 4, the user pushes the actuator bulb 48 with a finger. Pushing the actuator bulb 48 moves the dart 46 to pierce the foil inner seal 38. The active ingredient in the second component 44 mixes with the cosmetically acceptable carrier, the first component 18, to form the final composition in the reservoir 16. The user then removes the cap 8 from the spout 6 and pushes again on the actuator bulb 48 to dispense the mixed composition through the spout 6 for application and use.

Advantages of the invention are that the foil inner seal 38 maintains the integrity of the unstable ingredient in the second component 44 by keeping it separated from other ingredients/environments that could degrade it. The package is simple to activate and easy to use. The two components can be mixed to form the composition in minimal steps. Once mixed, the composition remains active for at least one week. The package 2 can be used with any suitable unstable active ingredient. Additionally, different active ingredients can be used with different suitable carriers, each in liquid or powder form so long as they are sufficiently fluid to allow mixing.

It is understood that various modifications and changes in the specific form and construction of the various parts can be made without departing from the scope of the following claims.

What is claimed is:

1. A fresh composition delivery system comprising:
a composition comprising:
a first component including a dermatologically acceptable carrier; and
a second component including at least one unstable active ingredient; and
a package for keeping the first component and second component of the composition separate before use, the package comprising:
a tube having a dispensing spout with a cap at a first end and an open second end, a first portion of the tube adjacent the first end defining a reservoir, a quantity of the first component stored in the reservoir, and a second portion of the tube adjacent the second end having an inner surface of the open second end of the tube defining an inwardly directed sealing surface;
a cartridge having a tubular body dimensioned to fit closely in the second portion of the tube, the body having an outwardly directed sealing surface for engaging the inwardly directed sealing surface of the tube in airtight engagement, an inner end of the body positioned in the tube such the inner end opens toward the reservoir, an outer end of the body opposite the inner end, a foil inner seal closing the inner end, an actuator membrane located in the body between the inner end and the outer end, the actuator membrane forming a hermetic barrier between the inner end and the outer end of the body, a chamber defined in the body between the foil inner seal and the actuator membrane, a quantity of the second component stored in the chamber, a dart mounted on the actuator membrane such that it projects into the chamber toward the foil inner seal, the actuator membrane movable toward the inner end such that the dart pierces the foil inner seal to release the quantity of the second component into the quantity of the first component to allow for mixing of the composition shortly before use;
wherein, the composition so formed by mixing of the first and second components in the reservoir has a level of the unstable active ingredient in the composition that decreases by less than 6% over 7 days when stored at 25° C. at 60% relative humidity.

2. The system of claim 1 wherein the actuator membrane is an elastomeric bulb projecting convexly toward the outer end.

3. The system of claim 1 further comprising a foil outer seal closing the outer end of the body of the cartridge.

4. The system of claim 3 wherein the outer foil seal has a tab projecting radially outwardly to facility removal of the foil outer seal from the outer end of the cartridge.

5. The system of claim 1 wherein the active ingredient is selected from one of Vitamin C, Vitamin E, oxidation susceptible botanicals, retinol, resveratrol, stilbenoids, tocopherols, retinoids, folic acid, hair dye, a caffeic, chlorogenic, or gallic acid, high polyunsaturated oils, high linolenic acid oils, flax seed oil, raspberry seed oil, cranberry seed oil, black current seed oil, Sysimbrium oil, Perilla seed oil, Camelina sativa oil, Salvia hispanica oil, high linoleic oils, pomegranate seed oil, Prunus ameniaca oil, Juglans regia oil, hemp seed oil or wheat germ oil.

6. The system of claim 1 wherein the active ingredient is in a powder form.

7. The system of claim 1 wherein a force required to move the actuator to advance the dart to pierce the inner foil seal is in the range of 5.6 lbs. to 7.2 lbs.

8. The system of claim 7 wherein the force required to move the actuator to advance the dart to pierce the inner foil seal is about 6.4 lbs.

9. The system of claim 2 wherein a force required to move the bulb to advance the dart to pierce the inner foil seal is in the range of 5.6 lbs. to 7.2 lbs.

10. The system of claim 9 wherein the force required to move the bulb to advance the dart to pierce the inner foil seal is about 6.4 lbs.
